(19) United States
(12) Patent Application Publication
(10) Pub. No.: US 2026/0217747 A1
(43) Pub. Date: Jul. 30, 2026

(54) Synthesis of Fosfomycin Disodium

(71) Applicant: Interquim S.A. de C.V., Cuautitlán Izcalli (MX)

(72) Inventor: Arturo De La Rosa Barrales, Cuautitlán Izcalli (MX)

(21) Appl. No.: 19/146,212

(22) PCT Filed: Jan. 5, 2023
(86) PCT No.: PCT/MX2023/050002

§ 371 Date: Jul. 7, 2025

(51) Int. Cl. C07F 9/655

(52) U.S. Cl. CPC C07F 9/65505

(57) ABSTRACT

The present invention provides a method for preparing fosfomycin disodium using sodium hydroxide and levo-fosfomycin dextro-phenylethylamine salt as raw materials to prepare a high-purity sodium fosfomycin product.

### FIELD OF THE INVENTION

The present invention relates to a new process for the preparation and stabilization of the salt of (2R, cis)-1,2-epoxypropyl-sodium phosphonate (fosfomycin disodium).

### BACKGROUND

Fosfomycin is a molecule isolated from Streptomyces fradiae culture broths. It is a broad-spectrum antibiotic used for the treatment of urinary tract infections that inhibits cell wall peptidoglycan synthesis of gram-positive and gram-negative bacteria (Magn. Reson. Chem. 2015, 53, 454-459, Antimicrob. Agent. Chemother, 1981, 20, 393).

Fosfomycin has poor stability mainly due to the easy opening of the epoxide group, being sensitive to temperature and humidity. The sodium and calcium salts of fosfomycin are widely used in the medical and veterinary fields to inhibit the growth of gram-positive and gram-negative pathogenic bacteria.

The main dosage form of fosfomycin is fosfomycin sodium powder injection, which is suitable for instillation and intravenous injection. In addition, it does not require a skin sensitization test and can be used in a wide range of individuals. It is safer and more effective for clinical use. At present, fosfomycin sodium is suitable for respiratory tract infections, skin and soft tissue infections, intestinal infections, urinary tract infections, sepsis, meningitis, peritonitis, osteomyelitis, uterine adnexitis, intrauterine infection, pelvic inflammatory disease, etc. caused by sensitive bacteria. It can be used in combination with other antibiotics to treat severe infections caused by sensitive bacteria. It can also be used in combination with vancomycin to treat methicillin-resistant Staphylococcus aureus (MRSA) infections.

There are four main salts of fosfomycin, namely sodium; calcium; tromethamine; and benzylamine. In Europe, Japan and Southeast Asia, the sodium and calcium salts are the most widely used; in the United States, fosfomycin tromethamine dominates and is widely sold. Various dosage forms of the salts are produced, mainly injectables, tablets and powders. In general, dosage forms include tablets, capsules, powders, dry syrups, and intravenous and intramuscular injections.

Fosfomycin sodium has the advantages of broad antibacterial spectrum, unique curative effect, low toxicity, no side effects and sensitization. Its mechanism of action is to inhibit bacterial cell wall synthesis, which can be combined with a bacterial cell wall synthase to prevent bacteria from using related substances.

Fosfomycin sodium has synergistic effects when used together with other antibiotics and is effective against gram-positive and gram-negative bacteria. It is mainly used for urinary tract, skin and soft tissue, intestinal infections caused by sensitive Gram-negative bacteria. It is used as a drug against “superbugs”, so it has received a great deal of clinical attention.

The methods of preparation of fosfomycin sodium include the sodium methoxide method, the exchange resin method and the sodium hydroxide method.

The synthesis of fosfomycin sodium has been reported in patents CN 11023702; CN 108558947; CN 108558946; CN 109694389; CN 101759721, which show that the reaction proceeds by employing stoichiometric amounts of base (NaOH or EtONa) in the appropriate solvent, such as anhydrous ethanol, methanol or water.

In CN 108558946 disodium fosfomycin is prepared by mixing anhydrous ethanol and L-(−)-phosphomycin-(+)-α-phenethylamine salt in a dry reaction flask and, at a certain temperature, a solution of sodium ethoxide in ethanol is added dropwise and, after addition, the reaction is incubated, the temperature is brought below 0° C., filtered, washed with a small amount of frozen absolute ethanol and the solid is dried under vacuum to obtain high purity fosfomycin sodium.

CN2009102174488.8 discloses a method of preparing high purity fosfomycin sodium. It starts from levo-fosfomycin dextrophenethylamine as raw material, dissociates under the action of an aqueous sodium hydroxide solution and heats the fosfomycin to 35-40° C. The reaction is carried out under stirring for 1-2 hours, and then allowed to stand for 2-3 hours, medicinal charcoal is added to the sodium salt solution, stirred for 10-30 minutes, filtered by suction and the filtrate is collected to obtain a sodium salt filtrate; the salt is placed in the reaction vessel and placed in an ice water bath, the sodium salt filtrate is centrifuged and washed with absolute ethanol to obtain white crystals, which are dried under reduced pressure to obtain a white crystalline powder. The method uses a large amount of anhydrous ethanol; 15-20 tons of anhydrous ethanol are consumed per ton of product, and the ethanol contains a large amount of water after use, the ethanol is recovered by ternary azeotropic distillation to obtain anhydrous ethanol, which is high cost and energy-intensive. In addition, a certain amount of diols is produced during the concentration of the aqueous solution, and the quality of the product is difficult to guarantee.

The sodium methoxide method uses methanol as the solvent, uses the levo-fosfomycin salt dextrophenethylamine to react with sodium methoxide, and then crystallizes with acetone to obtain a monosodium salt content of 60% to 65% and a disodium salt content of 40% to 35%, as well as an appreciable amount of neutral fosfomycin sodium, but the phenethylamine generated is not only soluble in methanol and difficult to recover, but also reacts with acetone affecting the purity of the product.

### DETAILED DESCRIPTION OF THE INVENTION

The object of the present invention is to provide a process for obtaining fosfomycin sodium from fosfomycin 1-phenylethylamine (FFEA) that complies with the quality parameters required by the European Pharmacopoeia.

Based on the previously known procedures, the synthesis of disodium fosfomycin was proposed through the exchange of phenylethylammonium ion for sodium in fosfomycin phenylethylamine using sodium hydroxide in methanol, controlling the physicochemical conditions of the reaction.

In the synthesis process object of the present invention some factors that determine the purity and yield of the final product were identified, thus it was identified that the complete dissolution of sodium hydroxide in methanol directly affects the quality parameters such as pH and optical rotation, i.e., the stoichiometric ratio of base to FFEA must be maintained after filtering of the solution in the process.

The temperature of the reaction and crystallization of the product (8°±2° C.), avoids the formation of unknown impurities that must be below 0.05%, according to the specifications of the finished product. Extrapolation tests were carried out at low and high temperatures starting from room temperature, for short and long reaction periods, observing that the optimum reaction conditions are a temperature of 8±2° C. for 0.5 hr, compared to a temperature of 8±2° C. for 3 hr of reaction. It was observed that, at the indicated crystallization temperature, fosfomycin sodium still meets the quality criteria for the finished product. However, if the temperature is lower the yield decreases slightly, either because of inefficient temperature-driven ion exchange or because of the partial solubility that the sodium salt has at low temperature.

The addition of anhydrous ethanol in the crystallization stage was set at 30 to 40 minutes, which allows control of crystal formation, but the ethanol addition time does not affect the morphology of the crystal, which turns out to be a powder. However, a longer ethanol addition time to the reaction mixture does not presuppose an alteration of the quality attributes of the product, so the interval of 30 to 40 minutes is adequate. The only attribute that could be affected by increasing the process time is the formation of impurities, however, this can be ruled out by the test at 8±2° C. for 3 hrs, in which the phenylethylamine phosphazine was left in reaction with NaOH/MeOH solution for a time of 3 hours, keeping the temperature low, obtaining a crude product within specifications and with total unknown impurities below 0.01% and less than 0.05% for the individual ones determined by HPLC. There is no drawback of increasing the addition time of anhydrous EtOH in the plant if necessary.

The problem of the formation of a slight liquid layer on the top of the crystallized product results in the product being out of specification in water content. Fosfomycin sodium has the physicochemical property of being very hygroscopic. Considering the above, tests were carried out to squeeze fosfomycin sodium under vacuum with nitrogen flow for 30 minutes, in these tests the formation of the liquid layer on the product was not observed. Therefore, the use of nitrogen in this filtration stage will prevent the formation of the liquid layer and consequently the product from melting.

The above conditions were used in the synthetic route for obtaining fosfomycin disodium from fosfomycin phenylethylamine shown below:

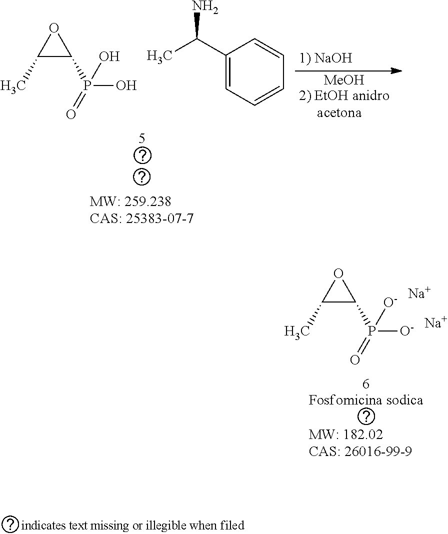

Three trials (A, B and C) of the process for obtaining disodium fosfomycin were carried out in which consistent results were obtained as evidenced in the tables of results shown below:

TABLE 1
Performance.

| Assay | Yield (% w/w) |
|---|---|
| A | 49 |
| B | 48 |
| C | 60 |

TABLE 2
Specifications

| Parameters | Specifications | Test A | Test B | Test C |
|---|---|---|---|---|
| Description | White or almost white | Complies | Complies | Complies |
| | powder, very hygroscopic. | | | |
| Water (KF) | <1.0% | Complies | Complies | Meets |
| | | 0.2% | 0.4% | 0.95% |
| Optical | From −13.0° to −15.0° | Meets −13.4° | Meets −13.4° | Meets −13.5° |
| rotation | calculated on anhydrous | | | |
| | basis | | | |
| IR | Meets | Meets | Meets | Complies |
| Appearance | Solution S is clear and no | Complies | Complies | Complies |
| of the solution | more intense in color than | | | |
| | reference solution B₉. | | | |
| pH | 9.0 a 10.5 | Meets 9.4 | Meets 9.4 | Meets 9.6 |
| Related substances | | | | |
| Determined by HPLC | | | | |
| Impurities A | ≤1.0% | Complies <LDD | Complies <LDDD | Complies <LDD |
| Unknown | ≤0.05% | Complies <LDD | Complies <LDDD | Complies <LDD |
| Impurities | | | | |
| Assay | Between 95.0 and 101.0% | Meets | Meets | Meets |
| | calculated on an anhydrous | 99.6% | 100.4% | 99.7% |
| | basis. | | | |
| Residual Solvents | | | | |
| Methanol | Not more than 3000 ppm | Complies | Complies | Complies |
| | | 24 ppm | 20 ppm | 68 ppm |
| Ethanol | Not more than 5000 ppm | Complies | Complies | Complies |
| | | 18 ppm | 12 ppm | 8 ppm |
| Acetone | Not more than 5000 ppm | Complies | Complies | Complies |
| | | 45 ppm | 46 ppm | 61 ppm |

### Process Description:

In the first stage of the process the sodium hydroxide solution is prepared as follows:

Methanol is cooled to a temperature of 12±2° C. with water/ice bath, three fractions every 5 minutes of sodium hydroxide are added under agitation, the final temperature reaches 20°-24° C. The bath is removed and kept under vigorous agitation at 20°-24° C. until complete dissolution of sodium hydroxide. The sodium hydroxide/methanol solution is filtered.

The second stage of the process consists of the reaction of sodium hydroxide with fosfomycin phenylethylamine, which includes the following steps:

The filtered sodium hydroxide solution is loaded directly into a reactor; the solution is cooled with vigorous stirring to a temperature of 12±2° C.; once this temperature is reached, the steady addition of fosfomycin phenylethylamine (FFEA) is started within 5 to 10 minutes; during the addition of FFEA, a temperature decrease to 6°-8° C. is observed; the mixture is kept in agitation for 30 minutes at 8°±2° C.; after this time anhydrous ethanol is added for 30 to 40 minutes at 8°±2° C.; the suspension is kept in agitation for an additional 30 minutes at 8°±2° C.; the suspension is filtered under nitrogen atmosphere and the cake is washed twice with anhydrous ethanol.

In the third stage, repulping is carried out as follows:

Anhydrous ethanol is charged into a reactor and brought to a temperature of 20°±2° C., then the solid obtained in the previous step is charged and kept under vigorous stirring for 30 minutes at 20°±2° C. The suspension is filtered under nitrogen atmosphere; two cake washes with anhydrous ethanol and two cake washes with acetone. The solid is dried under vacuum at a temperature of 35°±2° C. overnight. At the end of the drying time the product is weighed and packed under nitrogen atmosphere.

The disodium fosfomycin obtained is stored according to the following instructions:

The fosfomycin disodium salt is placed in a polyethylene bag and nitrogen is introduced; the bag is closed with a security band.

The bag of the product is placed inside a second polyethylene bag and bags of silica gel, as desiccant agent and nitrogen are introduced; the second bag is closed with a security band.

The second polyethylene bag is placed inside an aluminum bag and silica gel bags are inserted between the bags and nitrogen; the aluminum bag is sealed.

The yield of fosfomycin disodium obtained is between 48% to 60%, having a water content (KF) less than 1.0%; an optical rotation range between −13.4° and −13.5°; pH between 9.4 and 9.6; the residual solvents are below the detection limits by gas chromatography and the content of related substances is less than 0.1% and 0.05% for impurities A and unknown impurities respectively.

### Example: Obtaining Fosfomycin Disodium Using the Process Object of the Invention

Preparation of sodium hydroxide solution.

In a 2-liter flask of three openings provided with stoppers, thermometer, propeller and mechanical agitation, 700 mL of methanol are added.

The solvent is cooled to the temperature of 12°±2° C. with ice-water bath.

58 g of sodium hydroxide divided in three fractions are added, adding one fraction every 5 minutes. A final temperature of 20°−24° C. is reached.

The bath is removed and it is maintained in vigorous agitation to 20°-24° C. during one hour or until the complete dissolution of the sodium hydroxide. Filter to sodium hydroxide/methanol solution in a Büchner funnel.

Sodium hydroxide reaction with fosfomycin phenylethylamine.

The filtered basic solution is loaded directly into a 3-port reactor equipped with plugs, thermometer, propeller and mechanical stirring. The solution is cooled to a temperature of 12±2° C. with vigorous stirring. Once this temperature is reached, the constant addition of 200 g of fosfomycin phenylethylamine (FFEA) is started in a time of 5 to 10 minutes; during the addition of FFEA, a temperature decrease to 6°-8° C. is observed. It is kept in agitation for 30 minutes at 8°±2 C.°

800 mL of anhydrous ethanol is added over 30 to 40 minutes at 8°±2° C. The suspension is kept stirred for an additional 30 minutes at 8°±2 C.°

The suspension is filtered through a Büchner funnel. The cake is washed twice with anhydrous ethanol 0.5 volumes per wash.

### Repulping

In a reactor of three mouths of 3 liters provided with caps, thermometer, propeller and mechanical agitation 800 ml of anhydrous ethanol are loaded and it is taken to a temperature of 20°±2° C. The solid obtained in the previous stage is loaded in the reactor.

It is maintained in vigorous agitation for 30 minutes to 20°±2° C., the suspension is filtered in a Büchner funnel. Two washes are made to the cake with anhydrous ethanol, 100 mL for each wash (200 mL total) and two washes to the cake with acetone, 100 mL for each wash (200 mL total). The washes are done under nitrogen atmosphere.

The solid is dried under vacuum at a temperature of 35°±2° C. overnight. At the end of the drying time the product is weighed and bagged under nitrogen atmosphere.

### Storage

The fosfomycin disodium salt is placed in a polyethylene bag and nitrogen is introduced.

The bag is closed with a security band.

The product bag is placed inside another polyethylene bag.

Silica gel bags are placed as a desiccant agent and nitrogen is introduced.

The second bag is closed with a security band.

The second bag is placed inside an aluminum bag.

Silica gel bags are placed between the bags and nitrogen is introduced.

The aluminum bag is sealed.

1. Synthesis process for obtaining disodium fosfomycin, comprising the steps of:
a) preparing a homogeneous solution of sodium hydroxide in methanol at a temperature 12±2° C. with water/ice bath;
b) carrying out the reaction of the homogeneous solution of sodium hydroxide with fosfomycin phenylethylamine, at a temperature of 12±2° C. for 30 minutes;
c) adding anhydrous ethanol in a time of 30 to 40 minutes at a temperature of 8°±2° C.; and maintaining the suspension in agitation for an additional time of 30 minutes at 8°±2° C.; filtering the suspension to obtain a cake, the cake is washed twice with anhydrous ethanol under nitrogen atmosphere;
d) mixing the solid with anhydrous ethanol at a temperature of 20°±2° C., and it is maintained in vigorous agitation for 30 minutes at 20°±2° C.; filter the suspension under nitrogen atmosphere; make two washes to the cake with anhydrous ethanol and two washes to the cake with acetone;
e) drying the product under vacuum at a temperature of 35°±2° C. overnight; pack under nitrogen atmosphere; and
f) storing in polyethylene and aluminum bags with nitrogen and one or more drying agents.

2. The process according to claim 1, wherein the sodium hydroxide is added under agitation in three portions every 5 minutes.

3. The process according to claim 1, wherein the dissolution of the sodium hydroxide is complete and is filtered without leaving solid residues.

4. The process according to claim 1, wherein the addition of fosfomycin phenylethylamine is constant over a time of 5 to 10 minutes and is kept under agitation for an additional 30 minutes at 8°±2° C.; after this time anhydrous ethanol is added.

5. The process according to claim 4, wherein the addition of anhydrous ethanol is carried out for a time of 30 to 40 minutes at 8°±2° C. and, in addition, the suspension is kept in agitation for an additional 30 minutes at 8°±2° C.

6. The process according to claim 1, wherein the suspension of fosfomycin sodium and ethanol is filtered and washed twice with anhydrous ethanol under nitrogen atmosphere.

7. The process according to claim 6, wherein the fosfomycin sodium obtained in step c) is mixed with anhydrous ethanol and brought to a temperature of 20°±2° C., with vigorous stirring for 30 minutes at 20°±2° C.; the suspension is then filtered under nitrogen atmosphere; and washed twice with anhydrous ethanol and twice with acetone; the solid is dried under vacuum at a temperature of 35°±2° C. overnight.

8. The process according to claim 1, wherein the storage step comprises:
placing the fosfomycin disodium salt in a first polyethylene bag and introducing nitrogen; closing the bag;
placing the first polyethylene bag with the product inside a second polyethylene bag and introducing bags of a drying agent and nitrogen; closing the second bag;
place the second polyethylene bag inside an aluminum bag and introduce bags of a drying agent between the bags and nitrogen; seal the aluminum bag.

9. The process according to claim 8, wherein the desiccant agent is silica gel.

10. The process according to claim 1, wherein the yield of disodium fosfomycin obtained is between 48% and 60%, with a water content (KF) of less than 1.0%; an Optical Rotation range between −13.4° and −13.5°; pH between 9.4 and 9.6; the residual solvents are below the limits of detection by gas chromatography and the content of impurities A is less than 0.1% and the unknown impurities are less than 0.05%.

11. The process according to claim 1, wherein the purity of fosfomycin disodium obtained is between 99% and 100%.
